(19) United States
(12) Patent Application Publication
(10) Pub. No.: US 2025/0182322 A1
(43) Pub. Date: Jun. 5, 2025

(54) MACHINE LEARNING ENABLED LOCALIZATION OF FOVEAL CENTER IN SPECTRAL DOMAIN OPTICAL COHERENCE TOMOGRAPHY VOLUME SCANS

(71) Applicants: Genentech, Inc., South San Francisco, CA (US); Hoffmann-La Roche Inc., Little Falls, NJ (US)

(72) Inventors: Sharif Amit KAMRAN, Reno, NV (US); Michael Gregg KAWCZYNSKI, Nevada City, CA (US); Siva BALASUBRAMANIAN, San Francisco, CA (US); Andreas MAUNZ, Freiburg (DE)

(21) Appl. No.: 19/050,991

(22) Filed: Feb. 11, 2025

Related U.S. Application Data
(63) Continuation of application No. PCT/US2023/030181, filed on Aug. 14, 2023.
(60) Provisional application No. 63/371,297, filed on Aug. 12, 2022.

(51) Int. Cl. G06T 7/73; A61B 3/00; A61B 3/10; G06T 7/11

(52) U.S. Cl. CPC G06T 7/73; A61B 3/0025; A61B 3/102; G06T 7/11; G06T 2200/04; G06T 2207/10101; G06T 2207/20084; G06T 2207/30041

(57) ABSTRACT

A method and system for localizing a foveal center of a retina. An optical coherence tomography (OCT) volume for a retina of a subject is received. The OCT volume includes a plurality of OCT B-scans of the retina. A three-dimensional image input is generated for a model using the OCT volume. The model includes a three-dimensional convolutional neural network. The model is used to generate a foveal center position that includes three-dimensional coordinates for a foveal center of the retina based on the three-dimensional image input. The foveal center position may be used to generate an output that can be used in screening for retinal disease, diagnosing retinal disease, predicting treatment response, and/or managing retinal disease treatment.

### CROSS-REFERENCE TO RELATED APPLICATIONS

This application is a continuation of International Application No. PCT/US2023/030181, filed on Aug. 14, 2023, and entitled “Machine Learning Enabled Localization of Foveal Center in Spectral Domain Optical Coherence Tomography Volume Scans,” which is related to and claims the benefit of the priority date of U.S. Provisional Application No. 63/371,297, filed Aug. 12, 2022, and entitled “Machine Learning Enabled Localization of Foveal Center in Spectral Domain Optical Coherence Tomography Volume Scans,” each of which is incorporated herein by reference in its entirety.

### TECHNICAL FIELD

The subject matter described herein relates generally to optical coherence tomography (OCT) and more specifically to analysis methods and systems that use deep learning techniques to detect and localize the foveal center in OCT volumes.

### BACKGROUND

Various imaging techniques have been developed to capture medical images of tissues, which may then be analyzed to determine the presence or progression of diseases. For example, optical coherence tomography (OCT) refers to a technique where light waves are used to capture two-dimensional slice images (e.g., OCT B-scans) and three-dimensional volume images (e.g., OCT volumes) of tissues such as retinas of patients. OCT images can be analyzed and used to compute quantitative measurements for use in retinal disease screening, diagnosis, and treatment management. Such quantitative measurements may include measurements that are associated with a foveal center of the retina such as, for example, a central subfield thickness (CST) and other retinal thickness measurements (e.g., thickness measurements with respect to the Early Treatment Diabetic Retinopathy Study (ETDRS) grid). Thus, it may be desirable to have methods and systems that improve the accuracy and reliability of quantitative measurements computed based on the foveal center of retinas using OCT images.

### SUMMARY

In one or more embodiments, a method is provided in which an optical coherence tomography (OCT) volume for a retina of a subject is received. The OCT volume includes a plurality of OCT B-scans of the retina. A three-dimensional image input is generated for a model using the OCT volume. The model includes a three-dimensional convolutional neural network. The model is used to generate a foveal center position that includes three-dimensional coordinates for a foveal center of the retina based on the three-dimensional image input.

In one or more embodiments, a method is provided for training a model. The method includes, the method comprising receiving a training dataset that includes a plurality of optical coherence tomography (OCT) volumes for a plurality of retinas, wherein each of the plurality of OCT volumes includes a plurality of OCT B-scans. Training three-dimensional image input is generated for a model using the plurality of OCT volumes in the training dataset, the model comprising a three-dimensional convolutional neural network and a regression layer. The model is trained to generate a foveal center position comprising three-dimensional coordinates for a foveal center of a retina in a selected OCT volume based on the training three-dimensional image input.

In one or more embodiments, a system is provided that includes one or more data processors and a non-transitory computer readable storage medium containing instructions which, when executed on the one or more data processors, cause the one or more data processors to perform part or all of one or more methods disclosed herein. For example, the one or more data processors may be caused to receive an optical coherence tomography (OCT) volume for a retina of a subject, the optical coherence tomography (OCT) volume comprising a plurality of OCT B-scans of the retina; generate a three-dimensional image input for a model using the OCT volume, the model comprising a three-dimensional convolutional neural network; and generate, via the model, a foveal center position comprising three-dimensional coordinates for a foveal center of the retina based on the three-dimensional image input.

In one or more, a computer-program product is provided that is tangibly embodied in a non-transitory machine-readable storage medium and that includes instructions configured to cause one or more data processors to perform part or all of one or more methods disclosed herein. For example, the one or more data processors may be caused to receive an optical coherence tomography (OCT) volume for a retina of a subject, the optical coherence tomography (OCT) volume comprising a plurality of OCT B-scans of the retina; generate a three-dimensional image input for a model using the OCT volume, the model comprising a three-dimensional convolutional neural network; and generate, via the model, a foveal center position comprising three-dimensional coordinates for a foveal center of the retina based on the three-dimensional image input.

The terms and expressions which have been employed are used as terms of description and not of limitation, and there is no intention in the use of such terms and expressions of excluding any equivalents of the features shown and described or portions thereof, but it is recognized that various modifications are possible within the scope of the invention claimed. Thus, it should be understood that although the present invention as claimed has been specifically disclosed by embodiments and optional features, modification and variation of the concepts herein disclosed may be resorted to by those skilled in the art, and that such modifications and variations are considered to be within the scope of this invention as defined by the appended claims.

### BRIEF DESCRIPTION OF THE DRAWINGS

The accompanying drawings, which are incorporated in and constitute a part of this specification, show certain aspects of the subject matter disclosed herein and, together with the description, help explain some of the principles associated with the disclosed implementations.

FIG. 1 is a block diagram of an image processing system in accordance with one or more example embodiments.

FIG. 2 is a flowchart of a process for processing an optical coherence tomography (OCT) volume image of a retina of a subject in accordance with one or more example embodiments.

FIG. 3 is a flowchart of a process for training a model to generate a foveal center position in accordance with one or more embodiments.

FIG. 4 is an illustration of an example workflow for processing an OCT volume in accordance with one or more example embodiments.

FIG. 5 is a block diagram illustrating an example of a computing system, in accordance with one or more example embodiments.

It is to be understood that the figures are not necessarily drawn to scale, nor are the objects in the figures necessarily drawn to scale in relationship to one another. The figures are depictions that are intended to bring clarity and understanding to various embodiments of apparatuses, systems, and methods disclosed herein. Wherever possible, the same reference numbers will be used throughout the drawings to refer to the same or like parts. Moreover, it should be appreciated that the drawings are not intended to limit the scope of the present teachings in any way.

### DETAILED DESCRIPTION

### I. Overview

Medical imaging technologies are powerful tools that can be used to produce medical images that allow healthcare practitioners to better visualize and understand the medical issues of their patients, and as such provide the same more accurate diagnoses and treatment options. Certain parts of the retina, such as the fovea, may be imaged using these medical imaging techniques in order to accurately diagnose and treat retinal diseases, and monitor the retina more generally.

The fovea (or fovea centralis) is a small depression at the center of the macula lutea of the retina. The fovea is formed from densely packed cones and is surrounded by the parafovea belt, the perifovea outer region, and a larger peripheral area. The center of the fovea (or foveal center) is populated by the highest density of cones found in the retina while the density of cones is significantly lower in the perifovea. For example, the density of cones in the perifovea outer region is approximately 12 cones per 100 micrometers whereas approximately 50 cones occupy every 100 micrometers in the most central fovea. Although a relatively small portion of the retina, the fovea is responsible for acute central vision (e.g., foveal vision), which is integral for activities that rely on visual details. For instance, pointing the fovea at a certain direction may focus sensory processing resources on the most relevant sources of information.

The center of the fovea (or foveal center) is an important retinal feature for understanding disease state and vision loss. For example, as noted, the foveal center is populated with the highest density of cones found in the retina. A thickening of the retina, including swelling at or around the foveal center, is typically associated with a loss of vision acuity.

Accordingly, the center of the fovea or foveal center is a key landmark for generating further analyses of retinal features. locating the foveal center may be essential for measuring biomarkers relevant to diagnosing retinal diseases, evaluating disease burden, monitoring retinal disease progress, and predicting treatment response. For instance, central subfield thickness (CST), an important quantitative measurement for disease monitoring and treatment response, may be determined based on the foveal center of the retina. CST is typically measured by calculating an average retinal thickness across a circular area (e.g., a 1-millimeter ring) centered around the foveal center. Similarly, measurements relating to retinal segmentation may be based on the foveal center, such as, without limitations, generating the Early Treatment Diabetic Retinopathy Study (ETDRS) grid, or calculating internal limiting membrane (ILM) and/or Bruch's membrane (BM) boundary segmentations.

Optical coherence tomography (OCT) is a noninvasive imaging technique that is particularly popular for capturing images of the retina. OCT may be described as an ultrasonic scanning technique that scatters light waves from tissues to generate OCT images in the form of two-dimensional (2D) images and/or three-dimensional (3D) images of the tissues, similar to ultrasound scans that use sound waves to scan tissues. A 2D OCT image may also be referred to as an OCT slice, OCT cross-sectional image, or OCT scan (e.g., OCT B-scan). A 3D OCT image may be referred to as an OCT volume image and may be comprised of many OCT slice images.

For example, the presence of retinal disease in a patient, such as neovascular age-related macular degeneration, diabetic macular edema, or some other type of retinal disease, may cause a misalignment between the patient's foveal center and the geometric center of the resulting OCT volume. Similarly, poor subject fixation, eye movement, head tilting, the age of the subject, or a combination thereof may cause the foveal center of the subject to be offset relative to the geometric center of the resulting OCT volume. Poor fixation occurs when the subject's fixation on a target and the foveal center due not superimpose. Such poor fixation may be due to, for example, neurological conditions, eye tilting, subject age, etc. In some cases, there may be foveal center misalignment due to a lack of training or experience of the clinician performing the OCT scanning. For example, medical students in a teaching hospital may be less successful in aligning a subject's foveal center and the OCT geometric center relative to an experienced OCT clinician.

Currently, human graders make manual corrections for the foveal center to prevent further problems when using the foveal center to make quantitative measurements and identify biomarkers. But manual corrections can be time consuming, undesirable, and even unfeasible in large datasets. Further, manual corrections may not have the level of accuracy that is desired. For example, significant inter-clinician and intra-clinician variability can be present in subsequent realignment efforts. Further, these manual-based approaches to localizing the foveal center, which rely on expert annotations of optical coherence tomography (OCT) B-scans, may be resource-intensive and error-prone.

When an expert or manual grader is trying to detect the foveal center, the grader isolates and identifies the OCT B-scan of an OCT volume that most likely displays the foveal center, which is typically the middlemost or center OCT B-scan with respect to a transverse axis (e.g., each OCT B-scan of the OCT volume may be at a different position along on the transverse axis). On that selected OCT B-scan, the grader manually looks for the fovea on the OCT B-scan. For example, the grader may mark a lateral position of the foveal center and then infer an axial position of the foveal center. But the manually identified OCT B-scan may not be the correct OCT B-scan that contains the foveal center due to misalignment. The grader must then look at multiple OCT B-scans around the central OCT B-scan (with respect to transverse axis) to find the foveal center. Trying to find the foveal center in a large dataset containing hundreds or thousands of OCT volumes may be difficult. Further, in OCT images capturing retinas, the presence of pathology (or abnormalities) may alter the expected appearance of the fovea, making the detection of the fovea challenging even for experienced human graders.

Some currently available methods for localizing the foveal center use two-dimensional machine learning models (e.g., a two-dimensional convolutional neural network) to automatically detect a foveal center. Such a model is trained using a single OCT B-scan of the OCT volume for an iteration. This OCT B-scan is either the geometric center of the OCT volume or the OCT B-scan that has been selected by a human grader to correct for misalignment. But, as described above, this correction may not be accurate. Thus, training of a model based on such an OCT B-scan may lead to a model with reduced accuracy in detecting the foveal center. Further, these currently existing two-dimensional machine learning models use pixel-wise classification to detect the foveal center. Each pixel of an input OCT B-scan is assigned a classification (or probability) of whether that pixel is likely the foveal center or not. Post-processing resources are thus needed with these models to convert this pixel-wise classification for the various OCT B-scans of an OCT volume into simple coordinates for the foveal center. These types of techniques may thus be undesirable in many scenarios.

Thus, the embodiments described herein recognize that is important to have improved methods and systems for automatically detecting and localizing (e.g., identifying the coordinates for) a foveal center in an OCT volume accurately, precisely, and reliably. The embodiments described herein provide methods and systems for accurately, precisely, and reliably automatically detecting and generating three-dimensional coordinates for the foveal center. In one or more embodiments, an optical coherence tomography (OCT) volume for a retina of a subject is received. The OCT volume includes a plurality of OCT B-scans of the retina. A three-dimensional image input is generated for a model using the OCT volume. This model includes a three-dimensional convolutional neural network and a regression layer. The regression layer may be considered part of or separate from the three-dimensional convolutional neural network. Using the model, a foveal center position is generated. The foveal center position includes three-dimensional coordinates for a foveal center of the retina based on the three-dimensional image input. In other embodiments, the foveal center position includes two coordinates such as, for example, transverse and lateral coordinates. This foveal center position for the foveal center is generated with a greater level of accuracy than a manually-identified foveal center. Further, this foveal center position may be more accurate and reliable than a foveal center identified based on a pixel-wise classification of OCT B-scans.

Using the foveal center position generated by the model, a number of outputs may be generated. For example, quantitative measurements may be automatically computed from the OCT volume more accurately and reliably. Such quantitative measurements include, for example, without limitation, a central subfield thickness (CST) measurement and various retinal thickness measurements computed using a retinal grid. The retinal grid may be, for example, but is not limited, to the ETDRS grid.

In some embodiments, a model system is used that includes various modules or layers for automatically identifying a two-dimensional or three-dimensional foveal center position and additionally, automatically identifying a number of quantitative measurements (e.g., a CST measurement, one or more retinal thickness measurements for a retinal grid, etc.). The model system may also be used to automatically improve segmentation outputs such as the segmentation of various retinal layers or retinal features in an OCT volume (or in various OCT B-scans of the OCT volume). These segmentation outputs may have improved accuracy due to a more accurately identified foveal center position.

The embodiments described herein thus enable accurate and reliable foveal center localization in at least two dimensions. Because the embodiments described herein use a model to automatically and directly compute and output the coordinates of the foveal center in an OCT volume without requiring further processing, the amount of computing resources used may be reduced and the overall process may be made less labor-intensive. The foveal center localization further enables more accurate and reliable quantitative measurements and the generation of segmentation outputs, which in turn, may lead to improved retinal disease screening, diagnosis, management, treatment selection, treatment response prediction, treatment management, or a combination thereof.

### II. Example System for Foveal Center Detection

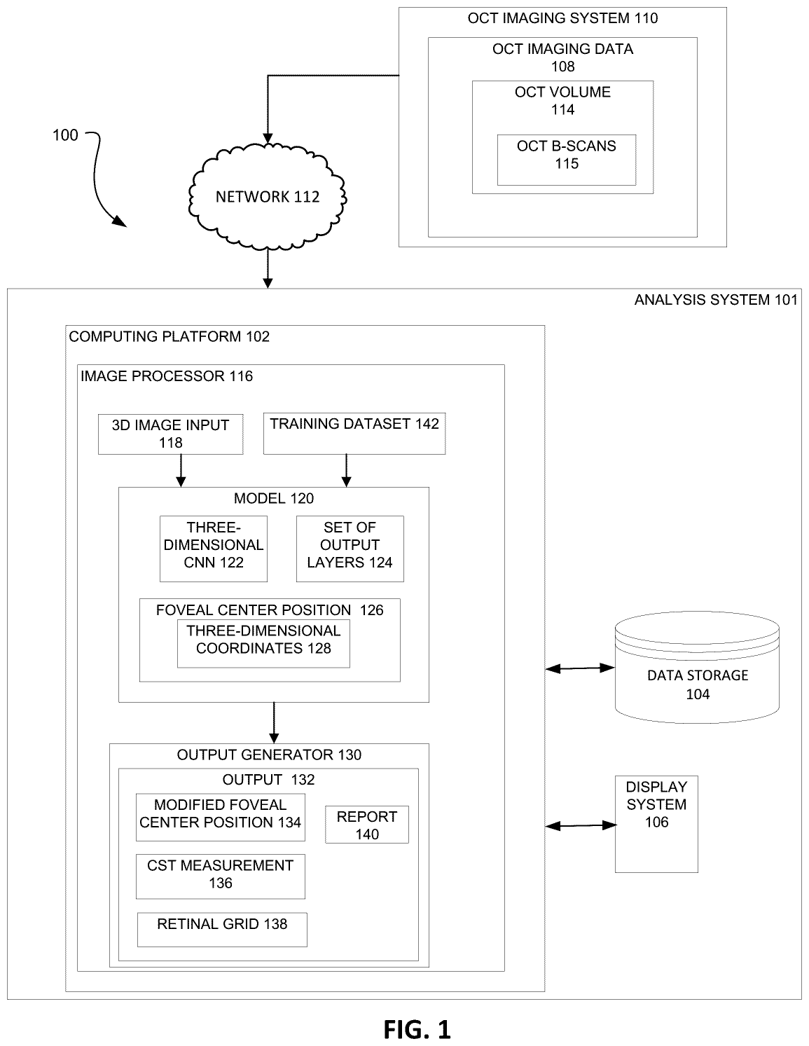

FIG. 1 is a block diagram of an image processing system 100 in accordance with one or more example embodiments. Image processing system 100 may be used to process ophthalmological images to extract features from such images, correct or otherwise adjust one or more features extracted from such images, segment such images, generates one or more outputs related to the diagnosis, screening, and/or treatment of an ophthalmological disorder, or a combination thereof.

The image processing system 100 includes analysis system 101. Analysis system 101 may be implemented using hardware, software, firmware, or a combination thereof. In one or more embodiments, analysis system 101 may include a computing platform 102, a data storage 104 (e.g., database, server, storage module, cloud storage, etc.), and a display system 106. Computing platform 102 may take various forms. In one or more embodiments, computing platform 102 includes a single computer (or computer system) or multiple computers in communication with each other. In other examples, computing platform 102 takes the form of a cloud computing platform, a mobile computing platform (e.g., laptop, a smartphone, a tablet, etc.), another processor-based device (e.g., a workstation or desktop computer) or a wearable computing device (e.g., a smartwatch), and/or the like or a combination thereof.

Data storage 104 and display system 106 are each in communication with computing platform 102. In some examples, data storage 104, display system 106, or both may be considered part of or otherwise integrated with computing platform 102. Thus, in some examples, computing platform 102, data storage 104, and display system 106 may be separate components in communication with each other, but in other examples, some combination of these components may be integrated together.

Computing platform 102 may be or may be part of a client device that is a processor-based device including, for example, a workstation, a desktop computer, a laptop computer, a smartphone, a tablet computer, a wearable apparatus, and/or the like.

The image processing system 100 may further include OCT imaging system 110, which may also be referred to an OCT scanner. OCT imaging system 110 may generate spectral domain (SD) OCT images. OCT imaging system 110 may generate OCT imaging data 108. OCT imaging data 108 may include any number of three-dimensional, two-dimensional, or one-dimensional OCT images. A three-dimensional OCT image may be referred to as an OCT volume. A two-dimensional OCT image may take the form of, for example, without limitation, an OCT B-scan.

In one or more embodiments, OCT imaging data 108 includes OCT volume 114 (e.g., SD-OCT volume) for a retina of a subject. OCT volume 114 may be comprised of a plurality of OCT B-scans 115 of the retina of the subject. The plurality of OCT B-scans 115 may include, for example, without limitation, 10s, 100s, 1000s, 10,000s, or some other number of OCT B-scans. An OCT B-scan may also be referred to as an OCT slice image or a cross-sectional OCT image.

In some embodiments, the retina is a healthy retina. In other embodiments, the retina is one that has been diagnosed with a retinal disease. For example, the diagnosis may be one of age-related macular degeneration (AMD), neovascular age-related macular degeneration (nAMD), diabetic retinopathy, macular edema, geographic atrophy, or some other type of retinal disease.

In one or more embodiments, the OCT imaging system 110 includes an optical coherence tomography (OCT) system (e.g., OCT scanner or machine) that is configured to generate OCT imaging data 108 for the tissue of a patient. For example, OCT imaging system 110 may be used to generate OCT imaging data 108 for the retina of a patient. In some instances, OCT imaging system 110 can be a large tabletop configuration used in clinical settings, a portable or handheld dedicated system, or a “smart” OCT system incorporated into user personal devices such as smartphones. In some cases, the OCT imaging system 110 may include an image denoiser that is configured to remove noise and other artifacts from a raw OCT volume image to generate the OCT volume 114.

Analysis system 101 may be in communication with OCT imaging system 110 via network 112. Network 112 may be implemented using a single network or multiple networks in combination. Network 112 may be implemented using any number of wired communications links, wireless communications links, optical communications links, or combination thereof. For example, in various embodiments, network 112 may include the Internet or one or more intranets, landline networks, wireless networks, and/or other appropriate types of networks. In another example, the network 112 may comprise a wireless telecommunications network (e.g., cellular phone network) adapted to communicate with other communication networks, such as the Internet. In some cases, network 112 includes at least one of a local area network (LAN), a virtual local area network (VLAN), a wide area network (WAN), a public land mobile network (PLMN), the Internet, or another type of network.

The OCT imaging system 110 and analysis system 101 may each include one or more electronic processors, electronic memories, and other appropriate electronic components for executing instructions such as program code and/or data stored on one or more computer readable mediums to implement the various applications, data, and steps described herein. For example, such instructions may be stored in one or more computer readable media such as memories or data storage devices (e.g., data storage 104) internal and/or external to various components of image processing system 100, and/or accessible over network 112.

Although only one of each of OCT imaging system 110 and the analysis system 101 is shown, there can be more than one of each in other embodiments. Further, although FIG. 1 shows the OCT imaging system 110 and the analysis system 101 as two separate components, in some embodiments, the OCT imaging system 110 and the analysis system 101 may be parts of the same system (e.g., and maintained by the same entity such as a health care provider or clinical trial administrator). In some cases, a portion of analysis system 101 may be implemented as part of OCT imaging system 110. For example, analysis system 101 may be configured to run as a module implemented using a processor, microprocessor, or some other hardware component of OCT imaging system 110. In still other embodiments, analysis system 101 may be implemented within a cloud computing system that can be accessed by or otherwise communicate with OCT imaging system 110.

The analysis system 101 may include an image processor 116 that is configured to receive OCT imaging data 108 from the OCT imaging system 110. The image processor 116 may be implemented using hardware, firmware, software, or a combination thereof. In one or more embodiments, image processor 116 may be implemented within computing platform 102. In some cases, at least a portion of (e.g., a module of) image processor 116 is implemented within OCT imaging system 110.

In one or more embodiments, image processor 116 may generate a three-dimensional (3D) image input 118 using OCT imaging data 108. For example, OCT volume 114 may be preprocessed using a set of preprocessing operations to form the 3D image input 118. The set of preprocessing operations may include, for example, without limitation, at least one of a normalization operation, a scaling operation, a resizing operation, a horizontal flipping operation, a vertical flipping operation, a cropping operation, a rotation operation, a noise filtering operation, or some other type of preprocessing operation. A normalization operation may be performed to normalize the coordinates of the coordinate system for OCT volume 114. In some cases, pixel values may be normalized (e.g., normalized to values between 0-1). A scaling operation may include, for example, scaling a coordinate system associated with the OCT volume 114. A resizing operation may include changing a size of each of the plurality of OCT B-scans 115. A preprocessing operation of the set of preprocessing operations may be performed on one or more of the plurality of OCT B-scans 115 of the OCT volume 114.

The image processor 116 may include a model 120, which may also be referred to as a foveal center model. Model 120 is a deep learning model that includes a three-dimensional convolutional neural network (CNN) 122 and set of output layers 124. The three-dimensional CNN 122 may include a fully convolutional neural network. Set of output layers 124 includes at least one regression layer. For example, with 3D image input 118 as its input, the three-dimensional CNN 122 may output spatial information. This spatial information may take the form of an output image indicating a position of the foveal center. The regression layer(s) is used to convert this output image into n-dimensional coordinates. The regression layer(s) may be considered part of or separate from the three-dimensional convolutional neural network.

In one or more embodiments, model 120 is a lightweight model. For example, the three-dimensional CNN 122 may be a lightweight convolutional neural network. The lightweight convolutional neural network is one in which computational complexity is reduced. This reduction may be performed in various ways including, for example, by reducing or removing dense layers.

Model 120 is used to detect and generate foveal center position 126 based on 3D image input 118. Foveal center position 122 includes three-dimensional coordinates 128 (i.e., three coordinates for three different axes of a coordinate system) for a foveal center of the retina. In one or more embodiments, the three-dimensional coordinates 128 may be for a selected coordinate system corresponding to OCT volume 114. For example, the three-dimensional coordinates 128 may include a transverse coordinate on a transverse axis, a lateral coordinate on a lateral axis, and an axial coordinate on an axial axis. The transverse axis may be the axis along which each of the plurality of OCT B-scans 115 lies. The lateral and axial axes may be the axes for the pixels of each of plurality of OCT B-scans 115. In some cases, each of the plurality of OCT B-scans may be indexed by (e.g., have an index corresponding to a value on) the transverse axis. The lateral axis may be, for example, a horizontal axis for each OCT B-scan. The axial axis may be a vertical axis for each OCT B-scan. In other embodiments, foveal center position 126 includes two coordinates for the foveal center. For example, foveal center position 126 may include a transverse coordinate and a lateral coordinate for the foveal center.

In some embodiments, model 120 may include a layer or module for rounding a transverse coordinate of three-dimensional coordinates 128 of foveal center position 126 to a value corresponding to an index associated with a particular OCT B-scan of plurality of OCT B-scans 115. For example, an initial value for a transverse coordinate of foveal center position 126 may be rounded to a rounded value that corresponds to an index associated with a particular OCT B-scan of the plurality of OCT B-scans 115. This rounded value forms one of three-dimensional coordinates 128 of foveal center position 126.

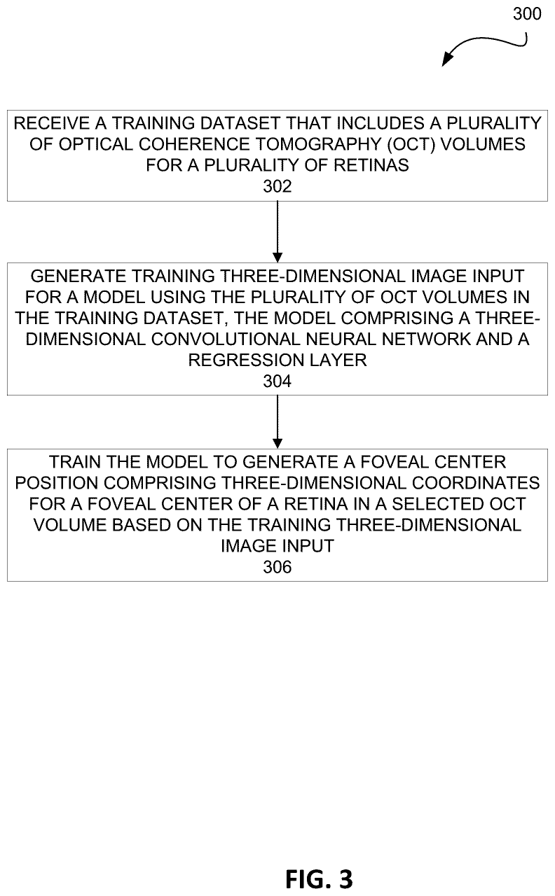

In one or more embodiments, model 120 may be trained with training dataset 142 to generate foveal center position 126. Training dataset 142 includes a plurality of OCT volumes. One or more OCT volumes of training dataset 142 may capture healthy retinas. One or more OCT volumes of training dataset 142 may capture retinas diagnosed with a retinal disease (e.g., AMD, nAMD, diabetic retinopathy, macular edema, geographic atrophy, or some other type of retinal disease). One example of a method for training model 120 (e.g., training three-dimensional CNN 122) may be described further below in FIG. 3.

Output generator 130 receives and processes foveal center position 126 to generate output 132 based on foveal center position 126. Output 132 may take various forms. For example, output 132 may include at least one of modified foveal center position 134, central subfield thickness (CST) measurement 136, retinal grid 138, a report 140 that identifies any one or more of the foveal center position 126, modified foveal center position 134, central subfield thickness (CST) measurement 136, retinal grid 138, or a combination thereof.

Output generator 130 may process foveal center position 126 to generate modified foveal center position 134, which may include, for example, coordinates for the foveal center that have been adjusted after transformation. For example, three-dimensional coordinates 128 may be transformed from a first selected coordinate system associated with OCT volume 114 to a second selected coordinate system. The second selected coordinate system may be associated with, for example, the retina or the subject (e.g., anatomical coordinate system). In some cases, modified foveal center position 134 may be generated by rounding one or more of three-dimensional coordinates 128 of foveal center position 126 to an integer or desired decimal level. In some cases, a transverse coordinate of three-dimensional coordinates 128 is rounded to a value corresponding to an index associated with a particular OCT B-scan of the plurality of OCT B-scans 115 to form modified foveal center position 134.

Output generator 130 may process foveal center position 126 to generate CST measurement 136, which may be one measure or indication of foveal thickness. The central subfield is a circular area that is 1 mm in diameter centered around the foveal center. CST measurement 136 is a measurement of the thickness of the macula in the central subfield. This thickness may be an average or mean thickness (e.g., with respect to a selected number of OCT B-scans around the foveal center). CST may also be referred to as central macular thickness or mean macular thickness.

Output generator 130 may process foveal center position 126 to determine (or otherwise define) retinal grid 138. Retinal grid 138 is a grid that divides the retina into regions based on the three-dimensional coordinates 128 of foveal center position 126. In one or more embodiments, retinal grid 138 takes the form of a retinal thickness grid. Such retinal thickness grids allow for quantitative measurements that may be important to screening for retinal disease, diagnosing retinal disease, monitoring disease progression, monitoring treatment response, selecting a treatment or treatment protocol, or a combination thereof. Such retinal thickness grids allow for these quantitative measurements to serve as biomarkers.

As one example, retinal grid 138 may be the Early Treatment Diabetic Retinopathy Study (ETDRS) grid. This grid divides the retina into nine regions—a central region, a set of four middle regions, and a center of four outer regions—that are centered based on the foveal center. Specifically, the central region is defined as the volume within a 1 mm diameter of the foveal center. The set of four middle regions are defined as four quadrants within the volume between the center region and a 3 mm diameter around the foveal center. The set of four outer regions are defined as four quadrants within the volume between the set of middle regions and a 6 mm diameter around the foveal center. Output generator 130 may use foveal center position 126—which is accurately generated—to define the dimensions of the ETDRS grid accurately. The quantitative measurements generated using an accurately defined ETDRS grid may improve capabilities with respect to screening, diagnosis, disease progression monitoring, treatment selection, treatment response prediction, treatment management, or a combination thereof.

In one or more embodiments, output generator 130 may generate report 140 that includes any of the above-identified outputs and/or other information. For example, report 140 may include a reproduction of or a modified version of the particular OCT B-scan containing the foveal center along with a graphical annotation or graphical label indicating foveal center position 126. For example, report 140 may modify OCT B-scan by at least one of resizing, flipping (horizontally, vertically, or both), cropping, rotating, reducing noise, adding graphical features to (e.g., adding one or more labels, colors, text, etc.), or otherwise modifying the OCT B-scan. In some embodiments, foveal center position 126 and/or modified foveal center position 134 may be identified on the reproduced or modified OCT B-can via text identifying the corresponding coordinates. In other cases, foveal center position 126 and/or modified foveal center position 134 may be identified by a pointer, a dot, a marker, or some other type of graphical feature.

In one or more embodiments, analysis system 101 stores OCT imaging data 108 obtained from OCT imaging system 110, 3D image input 118, foveal center position 126, output 132, other data generated during the processing of OCT imaging data 108, or a combination thereof, in data storage 104. In some embodiments, the portion of data storage 104 storing such information may be configured to comply with the security requirements of the Health Insurance Portability and Accountability (HIPAA) that mandate certain security procedures when handling patient data (e.g., such as OCT images of tissues of patients) (i.e., the data storage 104 may be HIPAA-compliant). For instance, the information being stored may be encrypted and anonymized. For example, the OCT volume 114 may be encrypted as well as processed to remove and/or obfuscate personally identifying information of the subjects from which the OCT volume 114 was obtained. In some instances, the communications link between OCT imaging system 110 and analysis system 101 that utilizes network 112 may also be HIPAA-compliant. For example, at least a portion of network 112 may be a virtual private network (VPN) that is end-to-end encrypted and configured to anonymize personally identifying information data transmitted therein.

Image processing system 100 may include any number or combination of servers and/or software components that operate to perform various processes related to the capturing and processing of OCT volumes of retinas. Examples of servers may include, for example, stand-alone and enterprise-class servers. In one or more embodiments, image processing system 100 may be operated and/or maintained by one or more different entities.

In some embodiments, OCT imaging system 110 may be maintained by an entity that is tasked with obtaining OCT imaging data 108 for tissue samples of subjects for the purposes of disease screening, diagnosis, disease monitoring, disease treatment, research, clinical trial management, or a combination thereof. For example, the entity may be a health care provider (e.g., ophthalmology healthcare provider) that seeks to obtain OCT imaging data 108 for retinas of subjects for use in diagnosing retinal diseases and/or other types of eye conditions. As another example, the entity may be an administrator of a clinical trial that is tasked with collecting OCT imaging data 108 for retinas of subjects to monitor retinal changes over the course of a disease, monitor treatment response, or both. Analysis system 101 may be maintained by a same or different entity (or entities) as OCT imaging system 110. For example, analysis system 101 may be maintained by an entity that is tasked with identifying or discovering biomarkers of retinal diseases from OCT images.

### III. Example Methodologies for Foveal Center Localization

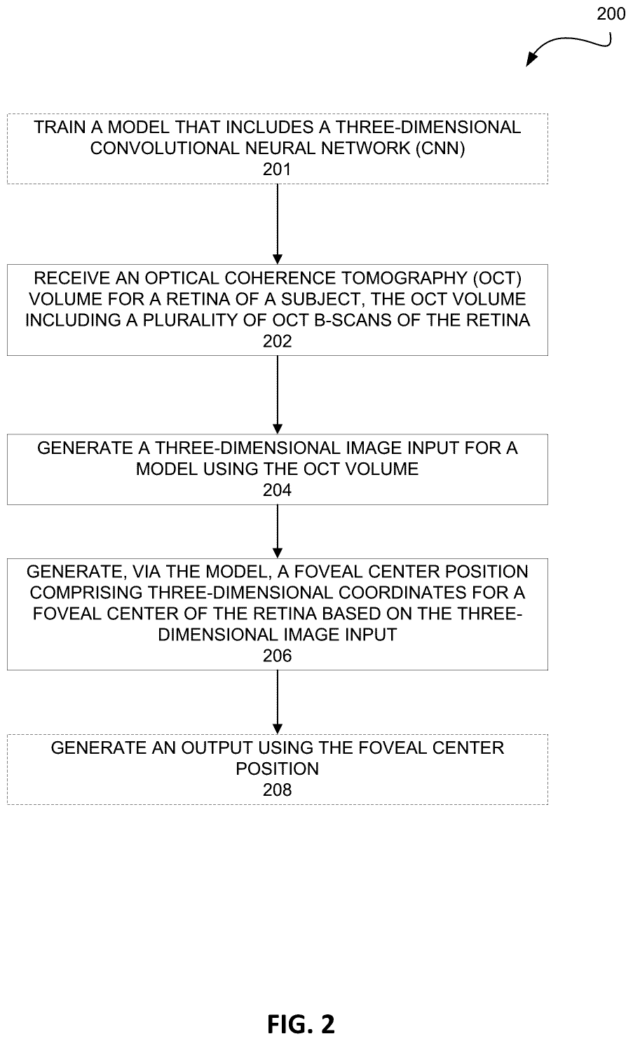

FIG. 2 is a flowchart of a process for processing an OCT volume of a retina of a subject in accordance with one or more example embodiments. Process 200 in FIG. 2 may be implemented using analysis system 101 in FIG. 1. In one or more embodiments, at least some of the steps of the process 200 may be performed by the processors of a computer or a server implemented as part of analysis system 101. It is understood that additional steps may be performed before, during, or after the steps of process 200 discussed below. In addition, in some embodiments, one or more of the steps may also be omitted or performed in different orders.

Process 200 may optionally include the step 201 of training a model that includes a three-dimensional convolutional neural network (CNN). The model may be, for example, model 120 in FIG. 1. The three-dimensional CNN may be implemented using, for example, three-dimensional CNN 122 in FIG. 1.

Step 202 of process 200 includes receiving an optical coherence tomography (OCT) volume for a retina of a subject, the OCT volume including a plurality of OCT B-scans of the retina. The OCT volume may be, for example, OCT volume 114 in FIG. 1. The plurality of OCT B-scans may be, for example, plurality of OCT B-scans 115 in FIG. 1. Each of the plurality of OCT B-scans is a cross-sectional view of the retina taken at a particular position with respect to a selected axis, which may be referred to as a transverse axis. Each OCT B-scan may have a horizontal axis (lateral axis) and a vertical axis (axial axis).

The retina may be healthy retina. Alternatively, the retina is one that has been diagnosed with or is suspected of having a retinal disease. The retinal disease may be, for example, age-related macular degeneration (AMD), neovascular age-related macular degeneration (nAMD), diabetic retinopathy, macular edema, geographic atrophy, or some other type of retinal disease.

Step 204 of process 200 includes generating a three-dimensional image input for a model using the OCT volume. The 3D image input may be, for example, 3D image input 118 in FIG. 1. The model may be, for example, model 120 in FIG. 1. The model includes a three-dimensional convolutional neural network and a regression layer at the output of the three-dimensional convolutional neural network. The regression layer may be considered part of or separate from the three-dimensional convolutional neural network. Step 204 may be performed in various ways. In one or more embodiments, generating the 3D image input includes performing a set of preprocessing operations on the OCT volume. The set of preprocessing operations may include, for example, at least one of a normalization operation, a scaling operation, a resizing operation, a horizontal flipping operation, a vertical flipping operation, a cropping operation, a rotation operation, a noise filtering operation, or some other type of preprocessing operation.

Step 206 includes generating, via the model, a foveal center position comprising three-dimensional coordinates for a foveal center of the retina based on the three-dimensional image input. The foveal center position may be, for example, foveal center position 126 that is comprised of three-dimensional coordinates 128 in FIG. 1. In one or more embodiments, the three-dimensional coordinates of the foveal center position include a transverse coordinate, an axial coordinate, and a lateral coordinate with respect to a selected coordinate system for the OCT volume. In some cases, the model generates the three-dimensional coordinates with values up to at least a selected decimal level. One or more of the three-dimensional coordinates may be rounded up or down to a selected decimal level. In some cases, one or more of the three-dimensional coordinates are rounded up or down to nearest integer. In some embodiments, an initial value for a transverse coordinate of the foveal center position is rounded to a rounded value that corresponds to an index associated with a particular OCT B-scan of the plurality of OCT B-scans. The rounded value becomes the transverse coordinate for the foveal center position.

Process 200 may optionally include step 208. Step 208 includes generating an output using the foveal center position. Step 208 may be performed using one or more sub-steps that are performed simultaneously, in sequence, or in some other type of combination. The output may be, for example, output 132 in FIG. 1. In one or more embodiments, the output includes a central subfield thickness measurement generated using the three-dimensional coordinates of the foveal center position. In some embodiments, the output includes a retinal grid that divides the retina into regions based on the three-dimensional coordinates of the foveal center position. The retinal grid may be, for example, an ETDRS grid that divides the retina into nine regions centered with respect to three-dimensional coordinates of the foveal center position.

In some embodiments, the output includes a modified foveal center position. For example, the foveal center position generated by the model in step 206 may be modified to form a modified foveal center position. In some cases, the three-dimensional coordinates of the foveal center position are transformed from a first selected coordinate system associated with the OCT volume to a second selected coordinate system associated with the retina or the subject (e.g., an anatomical coordinate system). In some cases, one or more of the three-dimensional coordinates are rounded up or down to a selected decimal level. In some cases, one or more of the three-dimensional coordinates are rounded up or down to a nearest integer. In an example embodiment, a transverse coordinate of the three-dimensional coordinates of the foveal center position is rounded to a value corresponding to an index associated with a particular OCT B-scan of the plurality of OCT B-scans of the retina.

In one or more embodiments, the output includes a transformation that is needed to shift the geometrical center of the OCT volume to the foveal center position. For example, the transformation may be a three-dimensional (or two-dimensional) shift that can be applied to the geometric center of the OCT volume to arrive at the foveal center position. This transformation can be applied to the foveal center identified by an OCT imaging system (e.g., OCT imaging system 110), which is at the geometric center of the OCT volume, so that the new foveal center will be correct when used for further analyses or processing.

In one or more embodiments, the output generated in optional step 208 includes a modified segmentation of the OCT volume. For example, the foveal center position may be used to modify a segmentation of at least one OCT B-scan of the plurality of OCT B-scans of the retina based on the three-dimensional coordinates of the foveal center position. In some cases, a segmented image output (e.g., a mask image output), which may be two-dimensional or three-dimensional, is modified based on the foveal center position.

In one or more embodiments, the output includes a report that includes any one or more of the outputs described above. In some cases, the report includes a reproduction of or a modified version of the particular OCT B-scan containing the foveal center along with a graphical annotation or graphical label indicating the foveal center position. For example, the report may modify an OCT B-scan by at least one of resizing, flipping (horizontally, vertically, or both), cropping, rotating, reducing noise, adding graphical features to (e.g., adding one or more labels, colors, text, etc.), or otherwise modifying the OCT B-scan. In some embodiments, the foveal center position and/or a modified foveal center position may be identified on the reproduced or modified OCT B-can via text identifying the corresponding coordinates. In other cases, the foveal center position and/or modified foveal center position may be identified by a pointer, a dot, a marker, or some other type of graphical feature.

Process 200, which may be implemented using image processing system 100 described in FIG. 1 or at least analysis system 101 in FIG. 1, provides an improvement to the technical field of retinal disease screening, diagnosis, and treatment management. For example, by improving the accuracy, precision, and reliability of foveal center localization, process 200 thereby improves the accuracy, precision, and reliability of quantitative measurements made based on the foveal center position, improves the identification of biomarkers for retinal disease and/or treatment response, and improves segmentation of OCT images. These improvements may be realized regardless of the type of OCT imaging system used to generate the OCT volume, the type of scanning protocol used to generate the OCT volume, the quality of the OCT volume, or a type of disrupting pathology or abnormality present in the retina. Accordingly, process 200 may facilitate improved automatic analysis of large datasets of OCT volumes even in the presence of varying conditions associated with the OCT volumes.

FIG. 3 is a flowchart of a process for training a model to generate a foveal center position in accordance with one or more embodiments. Process 300 in FIG. 3 may be implemented using analysis system 101 in FIG. 1. Process 300 may be one example of an implementation for step 201 of process 200 in FIG. 2. Further, it is understood that additional steps may be performed before, during, or after the steps of process 300 discussed below. In addition, in some embodiments, one or more of the steps may also be omitted or performed in different orders.

Step 302 of process 300 includes receiving a training dataset that includes a plurality of optical coherence tomography (OCT) volumes for a plurality of retinas. Each of the plurality of OCT volumes includes a plurality of OCT B-scans. The training dataset may be, for example, training dataset 142 in FIG. 1). The training dataset may include OCT volumes for retinas of varying health conditions. In one or more embodiments, the training dataset may include one or more OCT volumes for healthy retinas. In one or more embodiments, the training dataset may include OCT volumes for retinas diagnosed with a retinal disease such as AMD, nAMD, diabetic retinopathy, macular edema, geographic atrophy, or some other type of retinal disease. In some cases, the training data may include one rom ore OCT volumes for damaged retinas. The training dataset may include OCT volumes for a same type of retina (e.g., healthy or diseased or damaged) or different types of retinas. In some instances, the plurality of OCT volumes may be generated by more than one OCT imaging system (or type of OCT imaging system).

Step 304 includes generating training three-dimensional image input for a model using the plurality of OCT volumes in the training dataset, the model comprising a three-dimensional convolutional neural network and a regression layer. The regression layer may be considered part of or separate from the three-dimensional convolutional neural network. For example, step 304 may include performing a set of preprocessing operations on the plurality of OCT volumes to form the training three-dimensional image input. The set of preprocessing operations may include at least one of a normalization operation, a scaling operation, a resizing operation, a horizontal flipping operation, a vertical flipping operation, a cropping operation, a rotation operation, a noise filtering operation, or some other type of preprocessing operation.

Step 306 includes training the model to generate a foveal center position comprising three-dimensional coordinates for a foveal center of a retina in a selected OCT volume based on the training three-dimensional image input. The foveal center position may include a transverse coordinate, a lateral coordinate, and an axial coordinate for the foveal center in the OCT volume. The transverse coordinate may correspond to a particular OCT B-scan of the selected OCT volume. In some cases, the transverse coordinate may be a rounded value that corresponds to the particular OCT B-scan. In other cases, the transverse coordinate may be a value that can be further processed and rounded up or down to directly correspond with a particular OCT B-scan.

The trained model formed after step 306 may be, for example, model 120 in FIG. 1. Further, the trained model may be, for example, the model described with respect to process 200 in FIG. 2. The trained model may be used to accurately and reliably localize the foveal center such that other quantitative measurements may be accurately and reliably computed based on the foveal center position generated by the trained model. For example, the foveal center position may be used to generate an output such as output 132 described with respect to FIG. 1. The output may include a CST measurement and/or various retinal thickness measurements associated with a retinal grid (e.g., ETDRS grid). In one or more embodiments, the foveal center position can be used to improve retinal layer or fluid feature segmentation.

As described above, the training dataset used to train the model may include various types of OCT volumes. In one or more embodiments, the training dataset may include OCT volumes for retinas all diagnosed with a particular retinal disease (e.g., nAMD or diabetic macular edema). In some embodiments, the training dataset includes OCT volumes that are partitioned into training OCT volumes, validation OCT volumes, and test OCT volumes. In some embodiments, the OCT volumes of the training dataset are partitioned into only training OCT volumes and validation OCT volumes.

### IV. Example Workflow for Processing an OCT Volume

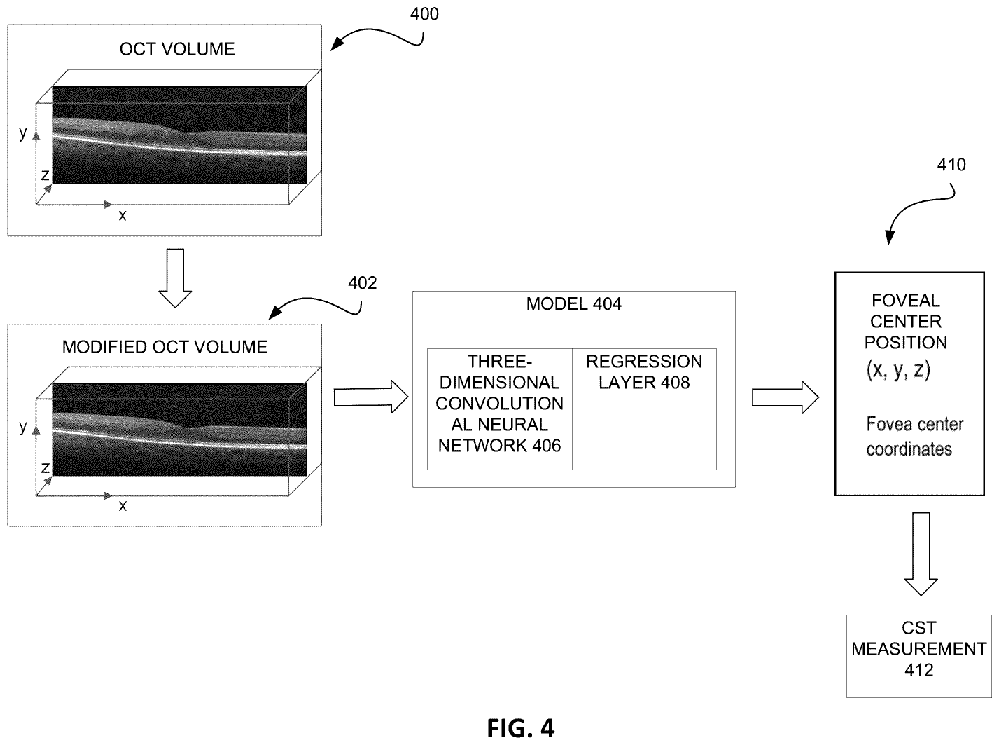

FIG. 4 is an illustration of an example workflow for processing an OCT volume in accordance with one or more example embodiments. OCT volume 400 may be one example of an implementation for OCT volume 114 in FIG. 1. OCT volume 400 has an x, y, and z coordinate system in which the x axis is the lateral axis, the y axis is the y axis, and the z axis is the transverse axis. Each of the OCT B-scans that make up the OCT volume 400 falls along, corresponds to, or may be indexed with a different coordinate on the transverse axis. OCT volume 400 may have a grayscale similar to that shown in FIG. 4. In other embodiments, some other type of grayscale or black-white scale may be used.

OCT volume 400 may be preprocessed to form modified OCT volume 402. Preprocessing may include, for example, but is not limited to, at least one of normalizing, scaling, resizing, horizontal flipping operation, vertical flipping, cropping, rotation, noise filtering, or some other type of preprocessing operation. In some examples, modified OCT volume 402 is created to ensure that the input sent into model 404 matches or is substantially similar in size, scale, and/or orientation to the types of OCT volumes on which model 404 was trained.

Model 404 may be one example of an implementation for model 120 in FIG. 1. Model 404 includes three-dimensional convolutional neural network 406 and regression layer 408. Three-dimensional convolutional neural network 406 may be one example of an implementation for three-dimensional convolutional neural network 122 in FIG. 1. Regression layer 408 may be one example of an implementation for a layer in set of output layers 124 in FIG. 1. Regression layer 408 may be considered part of or separate from the three-dimensional convolutional neural network 406. The three-dimensional convolutional neural network 406 of model 404 may be a fully convolutional neural network that includes convolutional layers. In one or more embodiments, model 404 includes one or more convolutional layers, one or more subsampling layers, and one or more fully connected layers.

In one or more embodiments, the hyper-parameters of model 404 include a batch size of 1 and an epoch of 60. Model 404 may be implemented using an AdamW optimizer. In one or more embodiments, model 404 may be specifically designed such that the total number of parameters used in model 404 is less than about 0.5 million parameters. Model 404 may be trained using a loss function. The loss function may be, for example, but is not limited to, mean absolute error (MAE), mean squared error (MSE), or some other type of loss function. Various metrics may be computed during the training and/or regular use of model 404. Such metrics include but are not limited to a flat R², a uniform R², a variance-weighted R², a raw R², and/or other types of metrics. In some embodiments, model 404 may be trained such that the output of three-dimensional convolutional neural network 406 is normalized (e.g., pixel values are normalized to a value between 0-1, etc.).

Model 404 processes modified OCT volume 402 to generate foveal center position 410. Foveal center position 410 includes three coordinates (x, y, and z coordinates for the foveal center). In other examples, foveal center position 410 includes two coordinates—x and z coordinates—for the foveal center. Foveal center position 410 may be one example of an implementation for foveal center position 126 described with respect to FIG. 1 and/or one example of an implementation for the foveal center position described with respect to process 200 in FIG. 2, process 300 in FIG. 3, or both.

In one or more embodiments, foveal center position 410 is used to generate a CST measurement 412. CST measurement may be one example of an implementation for CST measurement 134 described with respect to FIG. 1.

### V. Example Computing System

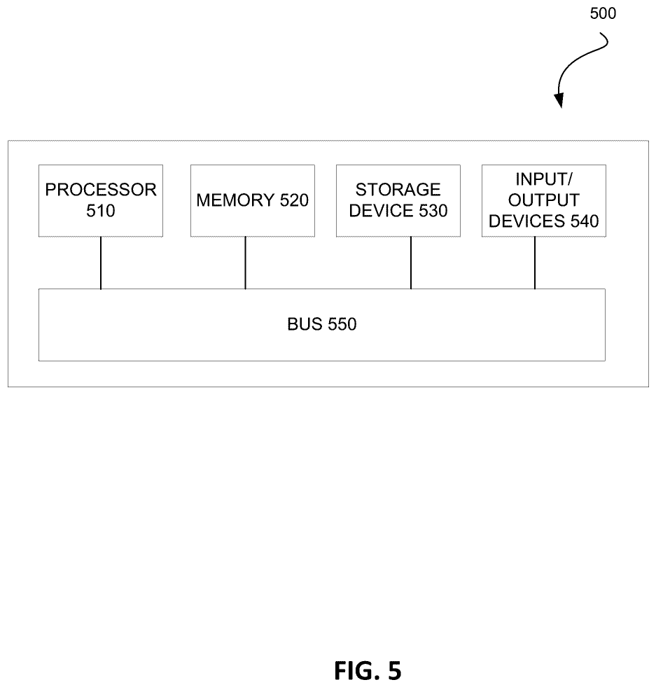

FIG. 5 is a block diagram illustrating an example of a computing system, in accordance with one or more example embodiments. Computing system 500 may be used to implement computing platform 102 in FIG. 1 and/or any components therein.

As shown in FIG. 5, the computing system 500 can include a processor 510, a memory 520, a storage device 530, and input/output devices 540. Computing system 500 may be one example implementation of analysis system 101 in FIG. 1. The processor 510, the memory 520, the storage device 530, and the input/output devices 540 can be interconnected via a system bus 550. The processor 510 is capable of processing instructions for execution within the computing system 500. Such executed instructions can implement one or more components of FIG. 5, for example, the analysis engine 520, the client device 530, and/or the like. In some example embodiments, the processor 3810 can be a single-threaded processor. Alternately, the processor 510 can be a multi-threaded processor. The processor 510 is capable of processing instructions stored in the memory 520 and/or on the storage device 530 to display graphical information for a user interface, such as display system 106 in FIG. 1.

The memory 520 is a computer readable medium such as volatile or non-volatile that stores information within the computing system 500. The memory 520 can store data structures representing configuration object databases, for example. The storage device 530 is capable of providing persistent storage for the computing system 500. The storage device 530 can be a floppy disk device, a hard disk device, an optical disk device, or a tape device, or other suitable persistent storage means. Storage device 530 may be one example implementation of data storage 104 in FIG. 1 The input/output device 540 provides input/output operations for the computing system 500. In some example embodiments, the input/output device 540 includes a keyboard and/or pointing device. In various implementations, the input/output device 540 includes a display unit for displaying graphical user interfaces.

According to some example embodiments, the input/output device 540 can provide input/output operations for a network device. For example, the input/output device 540 can include Ethernet ports or other networking ports to communicate with one or more wired and/or wireless networks (e.g., a local area network (LAN), a wide area network (WAN), the Internet).

In some example embodiments, the computing system 500 can be used to execute various interactive computer software applications that can be used for organization, analysis and/or storage of data in various formats. Alternatively, the computing system 500 can be used to execute any type of software applications. These applications can be used to perform various functionalities, e.g., planning functionalities (e.g., generating, managing, editing of spreadsheet documents, word processing documents, and/or any other objects, etc.), computing functionalities, communications functionalities, etc. The applications can include various add-in functionalities or can be standalone computing products and/or functionalities. Upon activation within the applications, the functionalities can be used to generate the user interface provided via the input/output device 540. The user interface can be generated and presented to a user by the computing system 500 (e.g., on a computer screen monitor, etc.).

One or more aspects or features of the subject matter described herein can be realized in digital electronic circuitry, integrated circuitry, specially designed ASICs, field programmable gate arrays (FPGAs) computer hardware, firmware, software, and/or combinations thereof. These various aspects or features can include implementation in one or more computer programs that are executable and/or interpretable on a programmable system including at least one programmable processor, which can be special or general purpose, coupled to receive data and instructions from, and to transmit data and instructions to, a storage system, at least one input device, and at least one output device. The programmable system or computing system may include clients and servers. A client and server are generally remote from each other and typically interact through a communication network. The relationship of client and server arises by virtue of computer programs running on the respective computers and having a client-server relationship to each other.

These computer programs, which can also be referred to as programs, software, software applications, applications, components, or code, include machine instructions for a programmable processor, and can be implemented in a high-level procedural and/or object-oriented programming language, and/or in assembly/machine language. As used herein, the term “machine-readable medium” refers to any computer program product, apparatus and/or device, such as for example magnetic discs, optical disks, memory, and Programmable Logic Devices (PLDs), used to provide machine instructions and/or data to a programmable processor, including a machine-readable medium that receives machine instructions as a machine-readable signal. The term “machine-readable signal” refers to any signal used to provide machine instructions and/or data to a programmable processor. The machine-readable medium can store such machine instructions non-transitorily, such as for example as would a non-transient solid-state memory or a magnetic hard drive or any equivalent storage medium. The machine-readable medium can alternatively or additionally store such machine instructions in a transient manner, such as for example, as would a processor cache or other random access memory associated with one or more physical processor cores.

To provide for interaction with a user, one or more aspects or features of the subject matter described herein can be implemented on a computer having a display device, such as for example a cathode ray tube (CRT) or a liquid crystal display (LCD) or a light emitting diode (LED) monitor for displaying information to the user and a keyboard and a pointing device, such as for example a mouse or a trackball, by which the user may provide input to the computer. Other kinds of devices can be used to provide for interaction with a user as well. For example, feedback provided to the user can be any form of sensory feedback, such as for example visual feedback, auditory feedback, or tactile feedback; and input from the user may be received in any form, including acoustic, speech, or tactile input. Other possible input devices include touch screens or other touch-sensitive devices such as single or multi-point resistive or capacitive track pads, voice recognition hardware and software, optical scanners, optical pointers, digital image capture devices and associated interpretation software, and the like.

### VI. Example Definitions and Context

The disclosure is not limited to these exemplary embodiments and applications or to the manner in which the exemplary embodiments and applications operate or are described herein. Moreover, the figures may show simplified or partial views, and the dimensions of elements in the figures may be exaggerated or otherwise not in proportion.

Unless otherwise defined, scientific and technical terms used in connection with the present teachings described herein shall have the meanings that are commonly understood by those of ordinary skill in the art. Further, unless otherwise required by context, singular terms shall include pluralities and plural terms shall include the singular. Generally, nomenclatures utilized in connection with, and techniques of, chemistry, biochemistry, molecular biology, pharmacology and toxicology are described herein are those well-known and commonly used in the art.

As used herein, “substantially” means sufficient to work for the intended purpose. The term “substantially” thus allows for minor, insignificant variations from an absolute or perfect state, dimension, measurement, result, or the like such as would be expected by a person of ordinary skill in the field but that do not appreciably affect overall performance. When used with respect to numerical values or parameters or characteristics that can be expressed as numerical values, “substantially” means within ten percent.

As used herein, the term “about” used with respect to numerical values or parameters or characteristics that can be expressed as numerical values means within ten percent of the numerical values. For example, “about 50” means a value in the range from 45 to 55, inclusive.

The term “ones” means more than one.

As used herein, the term “plurality” can be 2, 3, 4, 5, 6, 7, 8, 9, 10, or more.

As used herein, the term “set of” means one or more. For example, a set of items includes one or more items.

As used herein, the phrase “at least one of,” when used with a list of items, means different combinations of one or more of the listed items may be used and only one of the items in the list may be needed. The item may be a particular object, thing, step, operation, process, or category. In other words, “at least one of” means any combination of items or number of items may be used from the list, but not all of the items in the list may be required. For example, without limitation, “at least one of item A, item B, or item C” means item A; item A and item B; item B; item A, item B, and item C; item B and item C; or item A and C. In some cases, “at least one of item A, item B, or item C” means, but is not limited to, two of item A, one of item B, and ten of item C; four of item B and seven of item C; or some other suitable combination.

Where reference is made to a list of elements (e.g., elements a, b, c), such reference is intended to include any one of the listed elements by itself, any combination of less than all of the listed elements, and/or a combination of all of the listed elements.

The terms “one or more of A and B” and “and/or” may also occur in a list of two or more elements or features. Unless otherwise implicitly or explicitly contradicted by the context in which it used, such a phrase is intended to mean any of the listed elements or features individually or any of the recited elements or features in combination with any of the other recited elements or features. For example, the phrases and “one or more of A and B” and “A and/or B” are each intended to mean “A alone, B alone, or A and B together.” A similar interpretation is also intended for lists including three or more items. For example, the phrases “one or more of A, B, and C” and “A, B, and/or C” are each intended to mean “A alone, B alone, C alone, A and B together, A and C together, B and C together, or A and B and C together.”

The term “based on” is intended to mean “based at least in part on,” such that an unrecited feature or element is also permissible.

As used herein, a “model” includes at least one of an algorithm, a formula, a mathematical technique, a machine algorithm, a probability distribution or model, a model layer, a machine learning algorithm, or another type of mathematical or statistical representation.

The term “subject” may refer to a subject of a clinical trial, a person or animal undergoing treatment, a person or animal undergoing anti-cancer therapies, a person or animal being monitored for remission or recovery, a person or animal undergoing a preventative health analysis (e.g., due to their medical history), or any other person or patient or animal of interest. In various cases, “subject” and “patient” may be used interchangeably herein.

The term “OCT image” may refer to an image of a tissue, an organ, etc., such as a retina, that is scanned or captured using optical coherence tomography (OCT) imaging technology. The term may refer to one or both of 2D “slice” images and 3D “volume” images. When not explicitly indicated, the term may be understood to include OCT volume images.

As used herein, “machine learning” may include the practice of using algorithms to parse data, learn from it, and then make a determination or prediction about something in the world. Machine learning uses algorithms that can learn from data without relying on rules-based programming.

As used herein, an “artificial neural network” or “neural network” may refer to mathematical algorithms or computational models that mimic an interconnected group of artificial neurons that processes information based on a connectionistic approach to computation. Neural networks, which may also be referred to as neural nets, can employ one or more layers of nonlinear units to predict an output for a received input. Some neural networks include one or more hidden layers in addition to an output layer. The output of each hidden layer is used as input to the next layer in the network, i.e., the next hidden layer or the output layer. Each layer of the network generates an output from a received input in accordance with current values of a respective set of parameters. In the various embodiments, a reference to a “neural network” may be a reference to one or more neural networks.

A neural network may process information in, for example, two ways; when it is being trained (e.g., using a training dataset) it is in training mode and when it puts what it has learned into practice (e.g., using a test dataset) it is in inference (or prediction) mode. Neural networks may learn through a feedback process (e.g., backpropagation) which allows the network to adjust the weight factors (modifying its behavior) of the individual nodes in the intermediate hidden layers so that the output matches the outputs of the training data. In other words, a neural network may learn by being fed training data (learning examples) and eventually learns how to reach the correct output, even when it is presented with a new range or set of inputs.

### VII. Recitation of Example Embodiments

Embodiment 1. A method including receiving an optical coherence tomography (OCT) volume for a retina of a subject, the optical coherence tomography (OCT) volume including a plurality of OCT B-scans of the retina; generating a three-dimensional image input for a model using the OCT volume, the model including a three-dimensional convolutional neural network; and generating, via the model, a foveal center position including three-dimensional coordinates for a foveal center of the retina based on the three-dimensional image input.

Embodiment 2. The method of embodiment 1, wherein the three-dimensional coordinates of the foveal center position include a transverse coordinate, an axial coordinate, and a lateral coordinate with respect to a selected coordinate system for the OCT volume.

Embodiment 3. The method of embodiment 2 or embodiment 3, further including: generating a central subfield thickness measurement using the three-dimensional coordinates of the foveal center position.

Embodiment 4. The method of any one of embodiments 1-3, further including: determining a retinal grid that divides the retina into regions based on the three-dimensional coordinates of the foveal center position.

Embodiment 5. The method of embodiment 4, wherein the retinal grid is an Early Treatment Diabetic Retinopathy Study (ETDRS) grid that divides the retina into nine regions centered with respect to the three-dimensional coordinates of the foveal center position.

Embodiment 6. The method of any one of embodiments 1-5, further including: modifying a segmentation of at least one OCT B-scan of the plurality of OCT B-scans of the retina based on the three-dimensional coordinates of the foveal center position.

Embodiment 7. The method of any one of embodiments 1-6, wherein the model further includes a regression layer that is used to convert an output of the three-dimensional convolutional neural network into the three-dimensional coordinates.

Embodiment 8. The method of any one of embodiments 1-7, wherein generating the three-dimensional image input includes: performing a set of preprocessing operations on the OCT volume to form the three-dimensional image input, the set of preprocessing operations including at least one of a normalization operation, a scaling operation, a resizing operation, a horizontal flipping operation, a vertical flipping operation, a cropping operation, a rotation operation, or a noise filtering operation.

Embodiment 9. The method of any one of embodiments 1-8, further including: transforming the three-dimensional coordinates of the foveal center position from a first selected coordinate system associated with the OCT volume to a second selected coordinate system associated with the retina or the subject.

Embodiment 10. The method of any one of embodiments 1-9, wherein the retina of the subject is a healthy retina.

Embodiment 11. The method of any one of embodiments 1-9, wherein the retina of the subject is diagnosed with age-related macular degeneration (AMD), neovascular age-related macular degeneration (nAMD), diabetic retinopathy, macular edema, or geographic atrophy.

Embodiment 12. The method of any one of embodiments 1-11, wherein one coordinate of the three-dimensional coordinates of the foveal center position corresponds to a particular B-scan of the plurality of OCT B-scans of the retina.

Embodiment 13. The method of any one of embodiments 1-12, further including: rounding a transverse coordinate of the three-dimensional coordinates of the foveal center position to a value corresponding to an index associated with a particular OCT B-scan of the plurality of OCT B-scans of the retina.

Embodiment 14. The method of any one of embodiments 1-12, wherein generating, via the model including the three-dimensional convolutional neural network, the foveal center position includes rounding an initial value for a transverse coordinate of the foveal center position to a rounded value that corresponds to an index associated with a particular OCT B-scan of the plurality of OCT B-scans of the retina, wherein the rounded value is one of the three-dimensional coordinates of the foveal center position.

Embodiment 15. A method for training a model, the method including: receiving a training dataset that includes a plurality of optical coherence tomography (OCT) volumes for a plurality of retinas, wherein each of the plurality of OCT volumes includes a plurality of OCT B-scans; generating training three-dimensional image input for a model using the plurality of OCT volumes in the training dataset, the model including a three-dimensional convolutional neural network and a regression layer; and training the model to generate a foveal center position including three-dimensional coordinates for a foveal center of a retina in a selected OCT volume based on the training three-dimensional image input.

Embodiment 16. The method of embodiment 15, wherein the foveal center position includes a transverse coordinate, a lateral coordinate, and an axial coordinate for the foveal center in the OCT volume.

Embodiment 17. The method of embodiment 15 or embodiment 16, wherein generating the training three-dimensional image input includes: performing a set of preprocessing operations on the plurality of OCT volumes to form the training three-dimensional image input, the set of preprocessing operations including at least one of a normalization operation, a scaling operation, a resizing operation, a horizontal flipping operation, a vertical flipping operation, a cropping operation, a rotation operation, or a noise filtering operation.

Embodiment 18. The method of any one of embodiments 15-17, wherein the plurality of retinas includes at least one healthy retina.

Embodiment 19. The method of any one of embodiments 15-18, wherein the plurality of retinas includes at least one retina that is diagnosed with a retinal disease that is age-related macular degeneration (AMD), neovascular age-related macular degeneration (nAMD), diabetic retinopathy, macular edema, or geographic atrophy.

Embodiment 20. The method of any one of embodiments 15-19, wherein one coordinate of the three-dimensional coordinates of the foveal center position corresponds to a particular B-scan of the plurality of OCT B-scans of the retina.

Embodiment 21. A system that includes one or more data processors; and a non-transitory computer readable storage medium containing instructions which, when executed on the one or more data processors, cause the one or more data processors to: receive an optical coherence tomography (OCT) volume for a retina of a subject, the optical coherence tomography (OCT) volume including a plurality of OCT B-scans of the retina; generate a three-dimensional image input for a model using the OCT volume, the model including a three-dimensional convolutional neural network; and generate, via the model, a foveal center position including three-dimensional coordinates for a foveal center of the retina based on the three-dimensional image input.

Embodiment 22. A computer-program product tangibly embodied in a non-transitory machine-readable storage medium, including instructions configured to cause one or more data processors to: receive an optical coherence tomography (OCT) volume for a retina of a subject, the optical coherence tomography (OCT) volume including a plurality of OCT B-scans of the retina; generate a three-dimensional image input for a model using the OCT volume, the model including a three-dimensional convolutional neural network; and generate, via the model, a foveal center position including three-dimensional coordinates for a foveal center of the retina based on the three-dimensional image input.

Embodiment 23. A computer-program product tangibly embodied in a non-transitory machine-readable storage medium, including instructions configured to cause one or more data processors to perform the method of any one or more of embodiments 1-20.

Embodiment 24. A system that includes one or more data processors; and a non-transitory computer readable storage medium containing instructions which, when executed on the one or more data processors, cause the one or more data processors to perform the method of any one or more of embodiments 1-20.

### VIII. Additional Considerations

While the present teachings are described in conjunction with various embodiments, it is not intended that the present teachings be limited to such embodiments. On the contrary, the present teachings encompass various alternatives, modifications, and equivalents, as will be appreciated by those of skill in the art.

In describing the various embodiments, the specification may have presented a method and/or process as a particular sequence of steps. However, to the extent that the method or process does not rely on the particular order of steps set forth herein, the method or process should not be limited to the particular sequence of steps described, and one skilled in the art can readily appreciate that the sequences may be varied and still remain within the spirit and scope of the various embodiments.

Further, the subject matter described herein can be embodied in systems, apparatus, methods, and/or articles depending on the desired configuration. The implementations set forth in the description herein do not represent all implementations consistent with the subject matter described. Instead, they are merely some examples consistent with aspects related to the described subject matter. Although a few variations have been described in detail above, other modifications or additions are possible. In particular, further features and/or variations can be provided in addition to those set forth herein. For example, the implementations described above can be directed to various combinations and sub-combinations of the disclosed features and/or combinations and sub-combinations of several further features disclosed above. In addition, the logic flows depicted in the accompanying figures and/or described herein do not necessarily require the particular order shown, or sequential order, to achieve desirable results. Other implementations may be within the scope of the following claims.

What is claimed is:

1. A method comprising:
receiving an optical coherence tomography (OCT) volume for a retina of a subject, the optical coherence tomography (OCT) volume comprising a plurality of OCT B-scans of the retina;
generating a three-dimensional image input for a model using the OCT volume, the model comprising a three-dimensional convolutional neural network; and
generating, via the model, a foveal center position comprising three-dimensional coordinates for a foveal center of the retina based on the three-dimensional image input,
wherein the three-dimensional coordinates of the foveal center position include a transverse coordinate, an axial coordinate, and a lateral coordinate with respect to a selected coordinate system for the OCT volume.

2. The method of claim 1, further comprising:
generating a central subfield thickness measurement using the three-dimensional coordinates of the foveal center position.

3. The method of claim 1, further comprising:
determining a retinal grid that divides the retina into regions based on the three-dimensional coordinates of the foveal center position.

4. The method of claim 3, wherein the retinal grid is an Early Treatment Diabetic Retinopathy Study (ETDRS) grid that divides the retina into nine regions centered with respect to the three-dimensional coordinates of the foveal center position.

5. The method of claim 1, further comprising:
modifying a segmentation of at least one OCT B-scan of the plurality of OCT B-scans of the retina based on the three-dimensional coordinates of the foveal center position.

6. The method of claim 1, wherein the model further comprises a regression layer that is used to convert an output of the three-dimensional convolutional neural network into the three-dimensional coordinates.

7. The method of claim 1, wherein generating the three-dimensional image input comprises:
performing a set of preprocessing operations on the OCT volume to form the three-dimensional image input, the set of preprocessing operations including at least one of a normalization operation, a scaling operation, a resizing operation, a horizontal flipping operation, a vertical flipping operation, a cropping operation, a rotation operation, or a noise filtering operation.

8. The method of claim 1, further comprising:
transforming the three-dimensional coordinates of the foveal center position from a first selected coordinate system associated with the OCT volume to a second selected coordinate system associated with the retina or the subject.

9. The method of claim 1, wherein the retina of the subject is a healthy retina, or is diagnosed with age-related macular degeneration (AMD), neovascular age-related macular degeneration (nAMD), diabetic retinopathy, macular edema, or geographic atrophy.

10. The method of claim 1, wherein one coordinate of the three-dimensional coordinates of the foveal center position corresponds to a particular B-scan of the plurality of OCT B-scans of the retina.

11. The method of claim 1, further comprising:
rounding a transverse coordinate of the three-dimensional coordinates of the foveal center position to a value corresponding to an index associated with a particular OCT B-scan of the plurality of OCT B-scans of the retina.

12. The method of claim 1, wherein generating, via the model comprising the three-dimensional convolutional neural network, the foveal center position comprises:
rounding an initial value for a transverse coordinate of the foveal center position to a rounded value that corresponds to an index associated with a particular OCT B-scan of the plurality of OCT B-scans of the retina, wherein the rounded value is one of the three-dimensional coordinates of the foveal center position.

13. A method for training a model, the method comprising:
receiving a training dataset that includes a plurality of optical coherence tomography (OCT) volumes for a plurality of retinas, wherein each of the plurality of OCT volumes includes a plurality of OCT B-scans;
generating training three-dimensional image input for a model using the plurality of OCT volumes in the training dataset, the model comprising a three-dimensional convolutional neural network and a regression layer; and
training the model to generate a foveal center position comprising three-dimensional coordinates for a foveal center of a retina in a selected OCT volume based on the training three-dimensional image input,
wherein the three-dimensional coordinates of the foveal center position include a transverse coordinate, an axial coordinate, and a lateral coordinate for the foveal center in the OCT volume.

14. The method of claim 13, wherein generating the training three-dimensional image input comprises:
performing a set of preprocessing operations on the plurality of OCT volumes to form the training three-dimensional image input, the set of preprocessing operations including at least one of a normalization operation, a scaling operation, a resizing operation, a horizontal flipping operation, a vertical flipping operation, a cropping operation, a rotation operation, or a noise filtering operation.

15. The method of claim 13, wherein the plurality of retinas includes at least one healthy retina, or at least one retina that is diagnosed with a retinal disease that is age-related macular degeneration (AMD), neovascular age-related macular degeneration (nAMD), diabetic retinopathy, macular edema, or geographic atrophy.

16. The method of claim 13, wherein one coordinate of the three-dimensional coordinates of the foveal center position corresponds to a particular B-scan of the plurality of OCT B-scans of the retina.

17. A system comprising:
one or more data processors; and
a non-transitory computer readable storage medium containing instructions which, when executed on the one or more data processors, cause the one or more data processors to:
receive an optical coherence tomography (OCT) volume for a retina of a subject, the optical coherence tomography (OCT) volume comprising a plurality of OCT B-scans of the retina;
generate a three-dimensional image input for a model using the OCT volume, the model comprising a three-dimensional convolutional neural network; and
generate, via the model, a foveal center position comprising three-dimensional coordinates for a foveal center of the retina based on the three-dimensional image input,
wherein the three-dimensional coordinates of the foveal center position include a transverse coordinate, an axial coordinate, and a lateral coordinate with respect to a selected coordinate system for the OCT volume.

18. The system of claim 17, further comprising:
generating a central subfield thickness measurement using the three-dimensional coordinates of the foveal center position.

19. The system of claim 17, further comprising:
determining a retinal grid that divides the retina into regions based on the three-dimensional coordinates of the foveal center position.

20. The system of claim 17, further comprising:
modifying a segmentation of at least one OCT B-scan of the plurality of OCT B-scans of the retina based on the three-dimensional coordinates of the foveal center position.
